(19) United States
(12) Patent Application Publication
(10) Pub. No.: US 2006/0282112 A1
(43) Pub. Date: Dec. 14, 2006

(54) Method and apparatus for enhanced electrolytic detachment

(76) Inventor: Stephen Griffin, San Jose, CA (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/148,586

(22) Filed: Jun. 9, 2005

(51) Int. Cl. A61M 29/00

(52) U.S. Cl. 606/200

(57) ABSTRACT

A method and assembly for forming an occlusion within a vascular cavity. The assembly includes a catheter having an elongate shaft comprising a metallic tubular member extending from a proximal portion to a distal portion of the catheter, a delivery wire including an occlusion member detachably coupled thereto, and a power supply for providing an electrical current to initiate electrolysis at a sacrificial link between the delivery wire and the occlusion member in order to decouple the occlusion member from the delivery wire. The invention also includes a method of positioning and disposing an occlusion member within a vascular cavity using electrolysis, wherein a distal portion of the metallic tubular member serves as a first electrode and the sacrificial link serves as a second electrode. Thus, both the first electrode and the second electrode may be intravenously located proximate a vascular cavity.

### TECHNICAL FIELD

The invention relates to a method and assembly for forming an occlusion in a vascular cavity, such as veins, arteries, aneurysms, vascular malformations and arteriovenous fistulas. Some embodiments relate to a method and assembly for depositing an occlusion member within the interior of an intravascular aneurysm.

### BACKGROUND

Endovascular treatment of vascular cavities, such as intracranial aneurysms, has recently become an established therapeutic option. An aneurysm is an abnormal localized dilation of a vessel. Studies have found that unruptured intracranial aneurysms occur in about 3.6 to 6 percent of the population. If left untreated, an intracranial aneurysm may hemorrhage or rupture, causing permanent disability or even death. Untreated intracranial aneurysms may create a relatively high risk of mortality to a patient. One process of treating an intracranial aneurysm is by attempting to occlude the aneurysm with thrombogenic material. Occlusion of vascular cavities is typically through the use of detachable balloons, injectable glue, detachable or pushable coils, and injectable particles. Given the serious consequences of intracranial aneurismal rupture, there is an ongoing desire to provide improved methods and apparatus for treatment of vascular cavities, such as intracranial aneurysms.

### SUMMARY

One example embodiment is a method for forming an occlusion within a vascular cavity in a vasculature of a body of a patient. A catheter including a metallic tubular member may be advanced through the vasculature such that the distal portion of the metallic tubular member is proximate the vascular cavity and the proximal portion of the metallic tubular member is external the body. The metallic tubular member may extend a majority of the length of the catheter. An occlusion member detachably coupled to a metallic delivery wire at a sacrificial link may be advanced through the lumen of the catheter to the vascular cavity. The occlusion member may be positioned within the vascular cavity such that the distal portion of the metallic tubular member is proximate the sacrificial link located between the occlusion member and the delivery wire. An electrical current may be directed through the delivery wire and the metallic tubular member to initiate an electrolytic process to decouple the occlusion member from the delivery wire. During the electrolytic process, a first electrode may comprise a distal portion of the metallic tubular member and a second electrode may comprise the sacrificial link. The distal portion of the metallic tubular member serving as a first electrode may be a portion of the metallic tubular member in electrical communication with a bloodstream or other fluid medium within the anatomy of a patient. For example, the first electrode may be an exposed portion of the metallic tubular member in contact with a bloodstream, or the first electrode may be a distal portion of the metallic tubular member including an electrically conductive coating or covering. During electrolysis the anode, which may include the sacrificial link, may undergo oxidization and the cathode, which may include the distal portion of the metallic tubular member, may undergo reduction. Thus, during the electrolytic process the sacrificial link may be oxidized and dissipated such that the occlusion member is decoupled from the delivery wire.

Another example embodiment is an assembly for forming an occlusion within a vascular cavity in a vasculature of a body of a patient. The assembly includes a catheter having a proximal portion and a distal portion, wherein the catheter includes a metallic tubular member extending a majority of the length of the catheter. The metallic tubular member provides an electrically conductive pathway proximate a vascular cavity during use. An occlusion member detachably coupled to a delivery wire at a sacrificial link is disposed in the lumen of the catheter such that the sacrificial link is proximate the distal portion of the metallic tubular member. The delivery wire provides an electrically conductive pathway proximate a vascular cavity during use. A power source is coupled to the proximal portion of the metallic tubular member and the proximal portion of the delivery wire and provides an electrolytic current through the delivery wire to an anode comprising the sacrificial link and through the metallic tubular member to a cathode comprising a distal portion of the metallic tubular member in electrical communication with a bloodstream, or other fluid medium within the anatomy, thus forming an electrolytic cell within the vasculature proximate the vascular cavity that may function to detach the occlusion member from the delivery wire at the sacrificial link.

### BRIEF DESCRIPTION OF THE DRAWINGS

The invention may be more completely understood in consideration of the following detailed description of various embodiments in connection with the accompanying drawings, in which:

FIG. 1 is a plan view of an exemplary assembly for forming an occlusion in a vascular cavity within the scope of the invention;

FIG. 2 is a plan view of an exemplary delivery wire and occlusion member of the assembly within the scope of the invention;

FIG. 3 is an expanded view of the sacrificial link between the delivery wire and occlusion member illustrated in FIG. 2;

FIG. 4 is a plan view of an alternate embodiment of a delivery wire and occlusion member of the assembly within the scope of the invention;

FIG. 5 is a plan view of an exemplary catheter of the assembly within the scope of the invention;

FIG. 6 is a cross sectional view of the exemplary catheter of FIG. 5;

FIG. 7 is a cross sectional expanded view of proximal, intermediate, and distal regions of the exemplary catheter of FIG. 5;

FIG. 8 is a plan view of an exemplary navigational pathway through the vasculature of a body of a patient within the scope of the invention;

FIGS. 9-11 illustrate a method for depositing an occlusion member in a vascular cavity, such as an intravascular aneurysm, within the scope of the invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

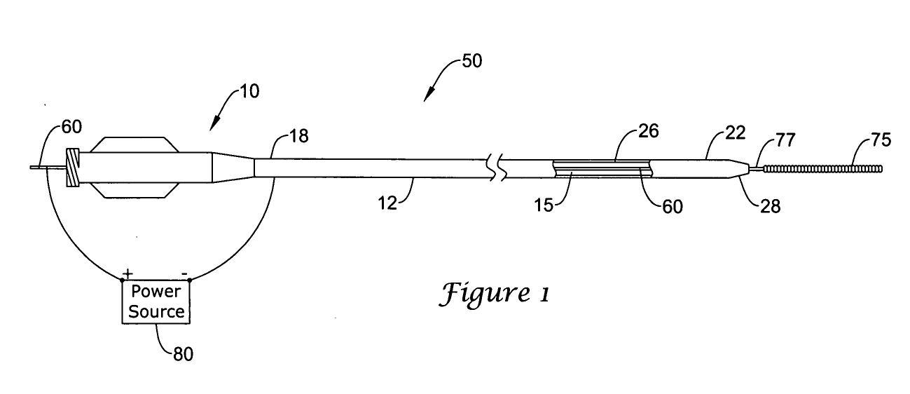

Refer now to FIG. 1, which illustrates a medical device assembly 50 in accordance with one example embodiment. Assembly 50 may include a catheter 10 comprising an elongate shaft 12 having a proximal portion 18 and a distal portion 22. Catheter 10 may include a manifold assembly 14 coupled to proximal portion 18 of elongate shaft 12. Distal portion 22 of elongate shaft 12 may include a distal tip 28. Elongate shaft 12 may comprise a metallic tubular member 26 extending from proximal portion 18 to distal portion 22 and defining a lumen 15 of catheter 10. Elongate shaft 12 of catheter 10 may have a length of about 20 cm or more, about 50 cm or more, about 75 cm or more, or about 100 cm or more.

Assembly 50 may further include a metallic delivery wire 60 disposed within and extending through lumen 15. An occlusion member 75, such as an embolic coil, may be coupled to a distal portion of delivery wire 60. Some exemplary embodiments of delivery wires and occlusion members within the scope of the invention are disclosed in U.S. Pat. Nos. 5,122,136; 5,354,295; 5,540,680; 5,569,245; 5,624,449; 5,911,717; 6,022,369; 6,346,091; and 6,463,317; all of which are herein incorporated by reference in their entirety. When occlusion member 75 is disposed within lumen 15 of catheter 10, occlusion member 75 may assume a longitudinally extended or straight configuration. When occlusion member 75 is disposed distal of distal end of catheter 10, and thus unconstrained by catheter 10, occlusion member 75 may be allowed to assume a coiled or twisting configuration and/or may be folded or urged in a desired arrangement.

A power supply 80 may be coupled to a proximal portion of delivery wire 60 and a proximal portion of metallic tubular member 26. Power supply 80, for example, may be a direct current power supply, an alternating current power supply, or a power supply switchable between a direct current and an alternating current. A positive terminal of a direct current power supply, as shown in FIG. 1, may be coupled to the proximal portion of the delivery wire 60 and a negative terminal of a direct current power supply may be coupled to the proximal portion of the metallic tubular member 26. Power supply 80 may provide a current through the assembly 50 to initiate an electrolytic process during use of the assembly in a fluid medium such as a bloodstream, which may be used as an electrolyte. A power supply, such as an alternating or direct current power supply, may additionally be used to initiate an electrothrombosis process.

An electrical current may be passed through the delivery wire 60 and the metallic tubular member 26 to initiate an electrolytic process at a coupling point between occlusion member 75 and a distal portion of delivery wire 60 while submerged in a conductive fluid medium such as a bloodstream of a vessel. A distal portion of metallic tubular member 26 may act as a first electrode and sacrificial link 77 between occlusion member 75 and delivery wire 60 may act as a second electrode during the process. For example, the first electrode may act as a cathode and the second electrode may act as an anode. The bloodstream may act as a conducting fluid, completing the electrolytic circuit between the anode and cathode. During electrolysis, the sacrificial link 77, or a portion thereof, may be oxidized and dissipated, thus decoupling the occlusion member 75 from the delivery wire 60. By positioning the distal portion of the metallic tubular member 26 proximate the sacrificial link 77, electrolytic resistivity between the sacrificial link 77 and the metallic tubular member 26 will be reduced. The distal portion of the metallic tubular member 26 may be placed intravascularly such that the distance between the sacrificial link 77 and the metallic tubular member 26 is less than 50 cm, less than 30 cm, less than 20 cm, less than 10 cm, less than 5 cm, less than 2 cm, or less than 1 cm. Therefore, time necessary for completion of the electrolytic process may be reduced and/or the power (i.e., voltage and/or current) transferred through the assembly during the electrolytic process may be reduced. Additionally, by placing the cathode intravascularly proximate the anode, the electrolytic current distributions are better controlled and localized, such as to reduce the possibility of stray thrombus formations occurring at unwanted sites or uncontrolled and possibly unwanted electrical current patterns being established elsewhere in the body.

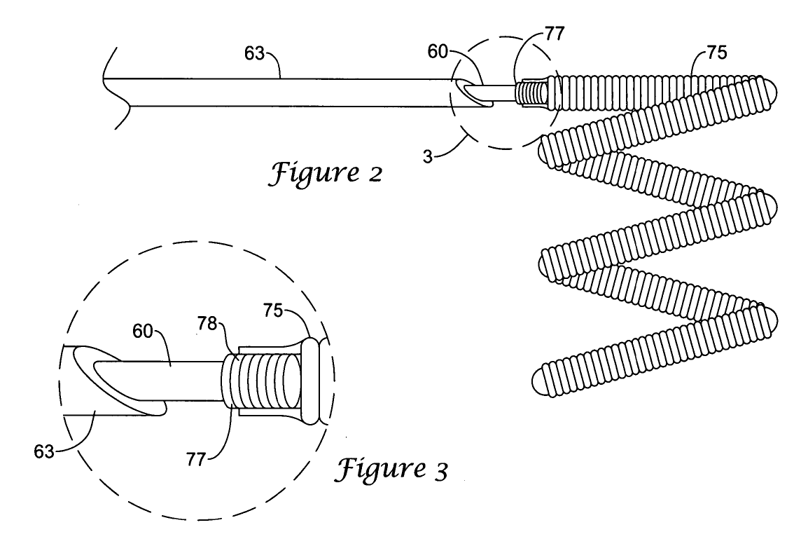

Now referring to FIGS. 2 and 3, an exemplary delivery wire 60 and occlusion member 75 will now be described. Delivery wire 60 may be referred to as a core wire and may be any suitable size or diameter. For example, delivery wire 60 may typically having a diameter of about 0.010 inch to about 0.020 inch (0.254-0.508 mm) and may typically have a length of about 50 cm to about 300 cm. Some examples of suitable materials for delivery wire 60 include metals, metal alloys, polymers, or the like, or combinations or mixtures thereof. For example, delivery wire 60 may comprise stainless steel, a stainless steel alloy, a nickel-titanium alloy such as nitinol, or any other material having a sufficient degree of conductivity. However, delivery wire 60 may comprise any of the materials discussed herein as being suitable for catheter 210. At least a portion of delivery wire 60 may be coated with a lubricious polymeric layer 63, thus reducing friction during delivery of an occlusion member and/or providing an insulative barrier to delivery wire 60. A distal portion of delivery wire 60 may be tapered to a smaller diameter. For example, the distal most 10 cm, 20 cm or 30 cm of the delivery wire 60 may be tapered to a smaller diameter.

An occlusion member 75, such as an embolic coil, may be detachably coupled to a distal portion of delivery wire 60. Occlusion member 75 may be about 1 to about 50 cm in length, and occlusion member 75 may have a sufficient flexibility such that the occlusion member is capable of deforming and folding and/or bending within a vascular cavity. Occlusion member 75 may be extremely pliable and its overall shape may be easily deformed. When inserted within a catheter, occlusion member 75 may be easily straightened to lie axially within the lumen of the catheter. Once disposed out of the distal tip of a catheter, occlusion member 75 may convert into a more shapely, nonlinear form such as shown in FIG. 2, and may be loosely deformed to the interior shape of a vascular cavity. Some examples of suitable materials for occlusion member 75 include metals, metal alloys, polymers, or the like, or combinations or mixtures thereof. For example, occlusion member 75 may comprise stainless steel, a stainless steel alloy, a nickel-titanium alloy such as nitinol, platinum, a platinum alloy, or the like. Platinum or a platinum alloy may be chosen due to the understanding that platinum is not generally subject to oxidation and dissipation during an electrolytic process. However, occlusion member 75 may comprise any of the materials discussed herein as being suitable for catheter 210. Occlusion member, or a portion thereof, may be coated with a thrombogenic agent, a drug or medication, a biological agent, or other coating.

Portions of delivery wire 60 and/or occlusion member 75 including an outer polymeric coating or covering 63, may not be affected by electrolysis. In other words, coated or insulated portions will not be oxidized and dissipated during an electrolytic process. For example, the sacrificial link 77 may remain exposed or otherwise be in electrical communication with a conductive fluid medium such as a bloodstream during use, thus initiating electrolysis in the region including the sacrificial link 77 during use. Another location along delivery wire 60 and/or occlusion member 75 may be left exposed, thus allowing electrolysis is this region.

FIG. 3 shows an enlarged view of an exemplary connection point or sacrificial link 77 between the occlusion member 75 and delivery wire 60. Sacrificial link 77 may be a discrete element disposed between occlusion member 75 and delivery wire 60. However, sacrificial link 77 may be a solder or weld, a portion of delivery wire, a portion of occlusion member 75, or any other portion of the assembly which may be oxidized and dissipated during an electrolytic process thereby decoupling occlusion member 75 from delivery wire 60. A sacrificial link 77, such as a coupling coil 78, may be coupled to a distal portion 61 of delivery wire 60. Coupling coil 78 may be soldered, welded, crimped, tacked, mechanically attached, friction fit, or otherwise coupled to delivery wire 60 as is known in the art. Coupling coil 78 may be any length. For example, coupling coil 78 may be about 3 cm to about 10 cm in length and may have any suitable diameter. For instance, coupling coil 78 may typically be between about 0.010 to about 0.020 inch (0.254-0.508 mm) in diameter. Coupling coil 78 may comprise any suitable material, for example stainless steel, a stainless steel alloy, or the like, or any other material which may be oxidized and dissipated during an electrolytic process. Occlusion member 75 may be coupled to coupling coil 78, such that coupling coil 78 is disposed between occlusion member 75 and delivery wire 60. Therefore, as coupling coil 78 is oxidized and dissipated during an electrolytic process, the occlusion member 75 may be decoupled from the delivery wire 60.

As shown in FIG. 3, delivery wire 60 may include a polymeric coating or covering 63 disposed about at least a portion of delivery wire 60 proximal of sacrificial link 77. Polymeric coating or covering 63 may enhance lubricity along at least a portion of delivery wire 60. Polymeric coating or covering 63 may terminate just proximal of sacrificial link 77. Thus, polymeric coating or covering 63 may provide an insulating layer to delivery wire 60 such that only an exposed portion of delivery wire 60 and/or coupling coil 78 is subjected to electrolysis. Sacrificial link 77 may be coated or covered with an electrically conductive material allowing an electrical current to pass through, or sacrificial link 77 may be coated or covered with a catalyst which may accelerate oxidation and dissipation of sacrificial link 77 during electrolysis.

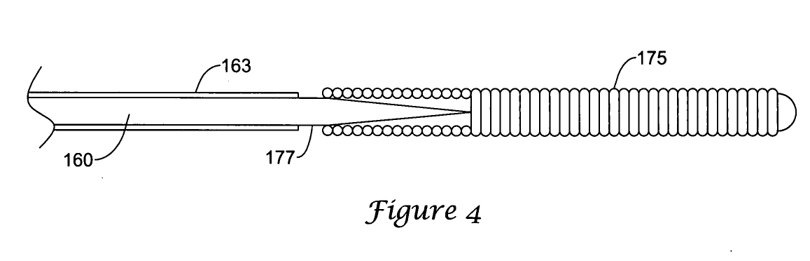

FIG. 4 shows an alternate embodiment of a delivery wire and occlusion member within the scope of the invention. Occlusion member 175 may be similar to occlusion member 75. For example, occlusion member 175 may be an extremely soft and flexible coil which may be easily deformed to pack the interior shape of a vascular cavity. Occlusion member 175 may comprise stainless steel, a stainless steel alloy, a nickel-titanium alloy such as nitinol, platinum, a platinum alloy, or other suitable material. Platinum or a platinum alloy may be chosen due to the understanding that platinum is not subject to oxidation and dissipation during an electrolytic process. Occlusion member 175 may be coupled to a distal portion of delivery wire 160 at a coupling point. The coupling point may be identified as a sacrificial link 177 similar to sacrificial link 77. Sacrificial link 177 is the region of the occlusion member/delivery wire assembly intended to be oxidized and dissipated during electrolysis, thus decoupling occlusion member 175 from delivery wire 160. Occlusion member 175 may be soldered, brazed, welded, crimped, mechanically coupled, or otherwise attached to a distal portion of delivery wire 160, or occlusion member 175 may be a distal extension of delivery wire 160.

Delivery wire 160 may include a polymeric covering or coating 163 disposed about at least a portion of delivery wire 160. Occlusion member 175 may additionally or alternatively include a covering or coating disposed about at least a portion of occlusion member 175. Polymeric covering or coating 163 may provide a layer of insulation to a portion of delivery wire 160 thus preventing electrolysis to the covered or coated portion.

The sacrificial link 177 may be exposed or otherwise configured to be subjected to oxidation during an electrolytic process. For example, occlusion member 175 may comprise a platinum or platinum alloy, or otherwise include a covering or coating, thus is not subject to oxidation and dissipation. Additionally or alternatively, a proximal portion of delivery wire 160 may include an insulative coating or covering 163, thus is not subject to oxidation and dissipation. During use, the sacrificial link 177 (which may be an exposed portion of delivery wire 160 as shown in FIG. 4) may be exposed to or otherwise be in electrical communication with a bloodstream of a vessel, or other fluid medium, thus defining a region for oxidization during an electrolytic process as discussed herein. Sacrificial link 177 may include a catalyst to accelerate oxidation and dissipation of sacrificial link 177 during electrolysis.

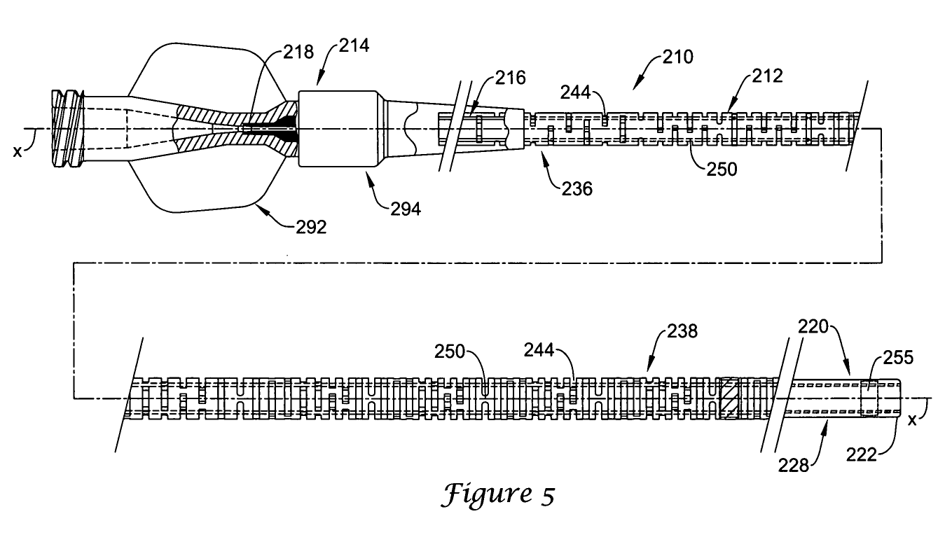

Referring to FIGS. 5-7, an exemplary catheter will be described. Catheter 210 may include an elongate shaft 212 having inner and outer tubular members 224/226. Either inner tubular member 224 or outer tubular member 226 may comprise metallic tubular member 26 of catheter 10, as discussed previously. The shaft 212 can be manufactured, include structure, and be made of materials so as to provide the desired characteristics of the catheter 210, depending upon the intended use. For example, the shaft 212 can be provided and/or manufactured so as to maintain a desired level of flexibility, torquability and/or other characteristics appropriate for maneuvering the catheter 210 as desired, for example, through the vasculature of a patient and/or proximate an intracranial aneurysm. As such, it should be understood that there is a broad range of possible shaft constructions that may be used—including those particularly discussed herein and others. Some other examples of suitable catheter shaft constructions and materials can be found in U.S. Pat. Nos. 5,569,218; 5,603,705; 5,674,208; 5,680,873; 5,733,248; 5,853,400; 5,860,963; 5,911,715; and 6,866,665, all of which are incorporated herein by reference. Some additional examples of shaft constructions include those disclosed in U.S. patent application Ser. No. 10/238,227 (Publication No. US-2004-0045645-A1), which is also incorporated herein by reference.

The shaft 12 may have a length and an outside diameter appropriate for its desired use, for example, to enable intravascular insertion and navigation. For example, in some embodiments, the shaft 212 may have a length in the range of about 1-300 centimeters or more, or in some embodiments in the range of about 20 cm-250 cm, and an outside diameter in the range of about 1 F to about 20 F, or in some embodiments, in the range of about 1 F to about 10 F. Additionally, although depicted as including a generally round outer diameter and a round cross-sectional shape, it can be appreciated that the shaft 212 can include other outer diameter and/or cross-sectional shapes or combinations of shapes without departing from the spirit of the invention. For example, the outer diameter and/or cross-sectional shape of the generally tubular shaft 212 may be oval, rectangular, square, triangular, polygonal, and the like, or combinations thereof, or any other suitable shape, depending upon the desired characteristics.

In some embodiments, the catheter 210 can be a microcatheter including a shaft 212 that is adapted and/or configured for use within small anatomies of the patient. For example, some embodiments are particularly useful in treating targets located in tortuous and/or narrow vessels. Some examples of such vessels may include those in the neurovascular system, or in certain sites within the coronary vascular system, or in sites within the peripheral vascular system such as superficial femoral, popliteal, or renal arteries. The target site in some embodiments is a neurovascular site, such as a site in the brain, which is accessible only via a tortuous vascular path, for example, a vascular path containing a plurality of bends or turns which may be greater than about 90° turns, and/or involving vessels which are in the range of about 8 mm or less, and in some cases as small as 2-3 mm or less, in diameter. As such, in some embodiments, the shaft 212 can include an outside diameter in the range of approximately 1 F-4 F.

However, in other embodiments, the catheter 210 may be used in other target sites within the anatomy of a patient, in which case the shaft 212 would be so adapted. For example, the catheter 210 may be suited for other uses in the digestive system, soft tissues, or any other use including insertion into an organism for medical uses, and the shaft 212 could be appropriately adapted for such uses. For example, in some embodiments, the catheter 210 may be used as an introducer sheath, in which case the shaft 212 may be significantly shorter. The catheter 210 may also include additional structure and materials adapted for a particular use and/or procedure. For example, in some other embodiments, the shaft 212 may include additional devices or structures such as inflation or anchoring members, device deployment members, sensors, optical elements, ablation devices, or the like, or any of a broad variety of other structures, depending upon the desired function and characteristics of the catheter 210.

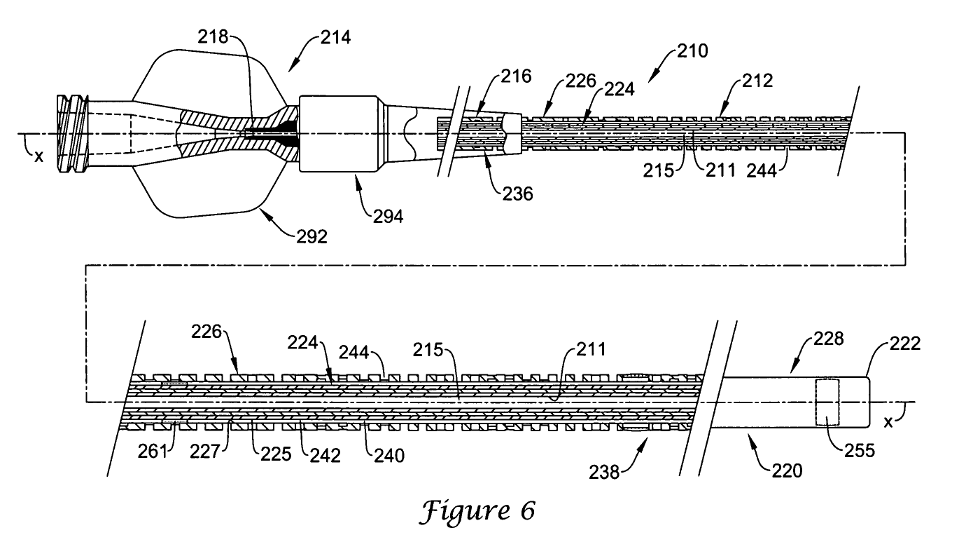

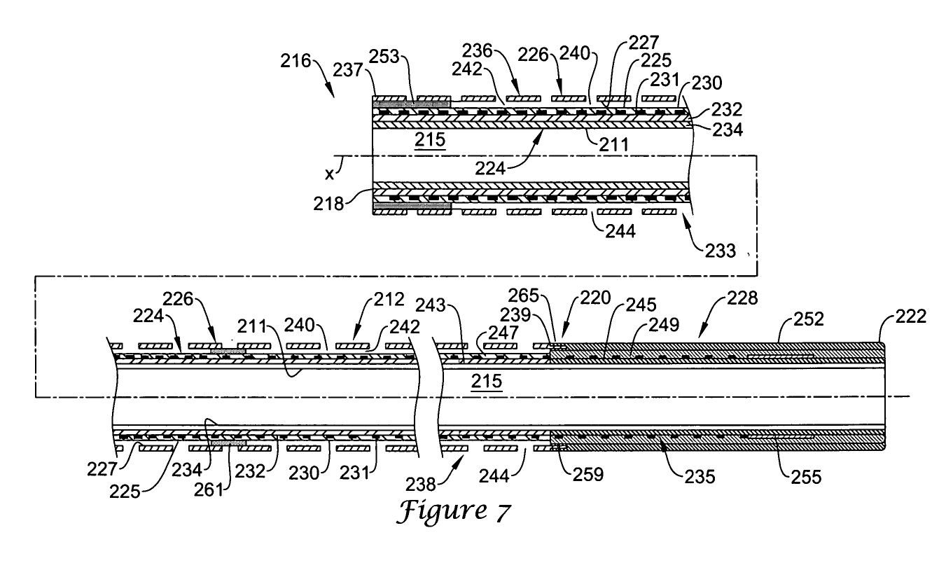

Referring now to FIG. 6, in at least some embodiments, the shaft 212 can have a generally tubular construction that includes at least one lumen 215 extending the length of the shaft 212 along the longitudinal axis x. This can also be seen with reference to FIG. 7, which is a partial cross-sectional view of the shaft 212 without the manifold assembly 214. The lumen 215 can be defined by an inner surface 211 of the shaft 212, and can have an inner diameter capable of transmitting fluids, or in some cases, receiving another medical device, such as a guidewire, a stent, a coil (such as an embolic coil, or the like), treatment particles (such as embolic particles, or the like), an ablation device, or another catheter, for example, a diagnostic catheter, a balloon catheter, a stent delivery catheter, or the like, or others. In some embodiments, the lumen 215 can be adapted and/or configured to accommodate another medical device having an outer diameter in the range of about 1 F to about 10 F.

In this embodiment, the shaft 212 includes a generally tubular construction including an inner tubular assembly and/or member 224, and an outer tubular assembly and/or member 226 disposed about at least a portion of the inner tubular member 224—however it should be understood that this is by way of example only. The inner tubular member 224 at least partially defines the inner surface 211 of the shaft 212, and thus defines the lumen 215.

The inner tubular member 224 can extend from a point within the distal portion 220 to a point within the proximal portion 216 of the shaft 212. The length of the inner tubular member 224 can vary, depending upon, for example, the length of the shaft 212, the desired characteristics and functions of the inner tubular member 224, and other such parameters. In some embodiments, the inner tubular member 224 can extend substantially the entire length of the shaft 212, for example, from a point adjacent the proximal end 218 to a point adjacent the distal end 222. For example, the length of the inner tubular member 224 can be in the range of about 1-300 centimeters or more, or in some embodiments in the range of about 20 cm-250 cm.

Referring to FIG. 7, the inner tubular member 224 can include a proximal portion 233 and a distal portion 235. The proximal and distal portions 233/235 can be any proximal or distal sections of the inner tubular member 224—however, in some cases the portions 233/235 can be defined with regard to the relative position of the inner and outer tubular members 224/226. For example, the distal portion 235 can be any portion of the inner tubular member 224 that extends distally beyond the distal end 239 of the outer tubular member 226, while the proximal portion 235 can be any portion of the inner tubular member 224 that is disposed within, or is proximal of a distal end 239 of the outer tubular member 226. In some embodiments, the distal portion 235 can have a length in the range of 0.5 cm or greater, or in the range of about 1 cm or greater, or 2 cm or greater, and in some embodiments in the range of about 3 to about 20 cm. In some embodiments, the distal portion 235 can be disposed within, and/or be apart of, or otherwise include a distal tip 228 construction, some examples of which will be discussed in more detail below.

The inner tubular member 224 may have an inner diameter, for example, defining the lumen 215, that is in the range of about 0.01 to about 0.05 inch in size, or in the range of about 0.015 to about 0.03 inch in size, or in the range of about 0.016 to about 0.026 inch in size. As indicated above, however, the lumen 215 (defined by the inner diameter of the inner tubular member 224) can be adapted and/or configured (e.g. sized) to accept other material, fluids, or medical devices, therein, and as such, the size of the lumen 215 can vary, depending upon the desired characteristics and intended use.

Additionally, the inner tubular member 224 can have an outer diameter that is in the range of about 0.011 to about 0.055 inch in size, or in the range of about 0.015 to about 0.03 inch in size, or in the range of about 0.019 to about 0.029 inch in size. It should be understood however, that these dimensions are provided by way of example embodiments only, and that in other embodiments, the size of the inner and outer diameter of the inner tubular member 224 can vary greatly from the dimensions given, depending upon the desired characteristics and function of the device.

The inner tubular member 224, or other portions of the shaft 212, may define one or more additional lumens depending upon the desired characteristics and function of the catheter 210, and such additional lumens can be shaped, size, adapted and/or configured the same as or different from lumen 215, depending upon the desired characteristic and functions.

The inner tubular member 224 may substantially overlay or cover the inner surface of the outer tubular member 226 throughout the length of the outer tubular member 226. However, in some embodiments at least a distal portion of the outer tubular member 226 may be free of the inner tubular member 224 such that a distal portion of the outer tubular member 226 is in electrical communication with fluid in the lumen 215. In other words, at least a portion of the inner surface 227 of the outer tubular member 226 may be exposed to the lumen 215 and remains unobstructed by any other layer of material, or at least a portion of the inner surface 227 may include an electrically conductive coating or covering. Thus, at least a portion of the inner surface 227 of outer tubular member 226 may be in electrical communication with a bloodstream or other fluid medium within lumen 215. For example, a portion of the inner surface 227 may be in fluid contact with a bloodstream when disposed in a vessel.

The inner tubular member 224 may include and/or be made of any of a broad variety of materials and/or structures. The inner tubular member 224 may have a single-layer tubular construction or a multi-layer tubular construction, or a combination thereof. For example, the inner tubular member 224 may be a single tubular member formed by a single layer of material, or in other embodiment, may be formed by a plurality of tubular members and/or a plurality of layers of material that may be the same and/or different, but in combination form the inner tubular member 224. In yet other embodiments, some portions of the inner tubular member 224 can include a single layer construction, while other portions may include a multi-layer construction. Some examples of suitable materials can include, but are not limited to, polymers, metals, metal alloys, or composites or combinations thereof.

Some examples of some suitable polymers can include, but are not limited to, polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), polyether-ester, some adhesive resin, such as modified polyolefin resin, polymer/metal composites, etc., or mixtures, blends or combinations thereof, and may also include or be made up of a lubricous polymer. Some other potentially suitable polymer materials may include those listed below with reference to the outer tubular member 226. One example of a suitable polyether block ester is available under the trade name ARNITEL, and one suitable example of a polyether block amide (PEBA) is available under the trade name PEBAX®, from ATOMCHEM POLYMERS, Birdsboro, Pa. In some embodiments, adhesive resins may be used, for example, as tie layers and/or as the material of the structures. One example of a suitable adhesive resin is a modified polyolefin resin available under the trade name ADMER®, from Mitsui Chemicals America, Inc. Additionally, polymer material can in some instances be blended with a liquid crystal polymer (LCP). For example, in some embodiments, the mixture can contain up to about 5% LCP. This has been found in some embodiments to enhance torqueability.

Some examples of suitable metals and metal alloys can include stainless steel, such as 304V, 304L, and 316L stainless steel; nickel-titanium alloy such as a superelastic (i.e. pseudoelastic) or linear elastic nitinol; nickel-chromium alloy; nickel-chromium-iron alloy; cobalt alloy; tungsten or tungsten alloys; tantalum or tantalum alloys, gold or gold alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); or the like; or other suitable metals, or combinations or alloys thereof. In some embodiments, it is desirable to use metals, or metal alloys that are suitable for metal joining techniques such as welding, soldering, brazing, crimping, friction fitting, adhesive bonding, etc.

Referring to FIG. 7, at least a portion of the inner tubular member 224 can have a multi-layer tubular construction. The example shown includes an inner layer 234, an intermediate layer 232 disposed about the inner layer 234, a reinforcing layer 231 disposed about the intermediate layer 232, and an outer layer 230 disposed about the reinforcing layer 231 and the intermediate layer 232. It should be understood that more or fewer layers can be used, with or without one or more reinforcing layers, depending upon the desired characteristics of the inner tubular member 224. Additionally, in other embodiments, the layers could be arranged differently to achieve desired properties. For example, the reinforcing layer 231 could be disposed at a different radial location, could be disposed entirely within another layer, could be disposed on the outer surface of the inner tubular member 224, or, as indicated above, could simply be absent. Furthermore, while the layers 230, 232 and 234 are described, these layers may be provided separately but form a single and/or unitary layer and/or structure. Some or all of the plurality of layers, for example layers 230, 231, 232, 234, may be made of any suitable material, for example, those discussed above for use in the inner tubular member 224.

In some embodiments, the inner layer 234 may include a lubricious polymer such as HDPE or PTFE, for example, or a copolymer of tetrafluoroethylene with perfluoroalkyl vinyl ether (PFA) (more specifically, perfluoropropyl vinyl ether or perfluoromethyl vinyl ether), or the like. In some particular embodiments, a PTFE tube is used as the inner layer 234, which can extend the length of the inner tubular member 224.

Furthermore, in some embodiments, the intermediate and outer layers 232/230 may each individually include a flexible polymer, for example a polymer material having a durometer in the range of about 5 D to about 90 D. For example, the intermediate and/or outer layers 232/230 can include or be made up of one or more tubular segments of a PEBA, a polyether-ester elastomer, or other like material. The durometer of the material used to form the intermediate and/or outer layers 232/230 may be the same, or may vary from one another—depending upon the characteristics desired. For example, the intermediate layer 232 may be made of a material having a higher durometer than the material of the outer layer 230 along at least a portion of the inner tubular member 224. In other embodiments, the reverse may be true, and in yet other embodiments, the two layers 230/232 may include the material having the same or similar flexibility characteristics.

In some embodiments, one or both of the layers 230/232 can be made up of a plurality of tubular segments including materials having different flexibility characteristics to impart varying degrees of flexibility to different longitudinal sections of the intermediate and/or outer layers 232/230. For example, in some embodiments, one or both of the layers 230/232 can include one or more proximal segments (e.g. 243/247) and one or more distal segments (e.g. 245/249). In some cases, the one or more proximal segments (e.g. 243/247) in either one or both layers 230/232 may include material having a higher durometer than the material included in the distal segment (e.g. 245/249) of each or both respective layer 230/232. Such a construction may be used, for example, to render a more distal portion of the inner tubular member 224 more flexible. Such an arrangement can also be helpful, for example, in providing a flexible distal tip construction, or a portion thereof.

For example, referring to the embodiment shown in FIG. 7, the intermediate layer 232 may include a proximal portion 243 including and/or made of a flexible polymer, such as a PEBA, a polyether-ester elastomer, or other like material, having a durometer in the range of about 40 D to about 70 D. The intermediate layer 232 may also include a distal portion 245 including and/or made of a flexible polymer having a durometer in the range of about 15 D to about 35 D. Additionally, the outer layer 230 may include a proximal portion 247 including and/or made of such a flexible polymer having a durometer in the range of about 25 D to about 55 D. The outer layer 230 may also include a distal portion 249 including and/or made of such a flexible polymer having a durometer in the range of about 15 D to about 35 D.

The one or more reinforcing layer 231, if present, can be constructed with any suitable materials and structures to impart the desired characteristics to the inner tubular member 224. The reinforcing layer 231 can include one or more support members that can comprise, for example, a braid, a coil, a filament or wire, or series of such structures, or the like, including material and/or structure adapted to provide the desired characteristics. Examples of suitable materials for constructing the reinforcing layer include polymers, metals, or metal alloys such as those discussed above, or the like, or any of a broad variety of other suitable material.

In some embodiments, the reinforcing layer 231 can be a coil 231. The coil 231 may be formed of an elongated filament (e.g. wire, ribbon, or the like) having appropriate dimension and shape to achieve the desired torque, flexibility, and/or other characteristic. For example, the filament used to form the coil 231 may be round, flat, oval, rectangular, square, triangle, polygonal, and the like, or any suitable shape. The coil 231 can be wrapped in a helical fashion by conventional winding techniques. The pitch of adjacent turns of coil 231 may be tightly wrapped so that each turn touches the succeeding turn, or the pitch may be set such that coil 231 is wrapped in an open fashion. The pitch can be constant throughout the length of the coil 231, or can vary, depending upon the desired characteristics, for example flexibility. For example, in some embodiments, the coil 231 can include a distal portion including a relatively open pitch, and a proximal portion having a relatively more closed pitch, such that the coil is more flexible in the distal portion than in the proximal portion. The reinforcing layer 231 may extend the entire length of the inner tubular member 224, or may extend only along a portion of the length thereof.

The inner tubular member 224 can be constructed using any one or a combination of appropriate methods and/or techniques, for example, extrusion, co-extrusion, interrupted layer co-extrusion (ILC), heat bonding techniques, heat shrink techniques, fusing, winding, disposing, adhesive bonding, mechanical bonding, soldering, welding, molding, casting, or the like, or others. In some embodiments, one or more of the layers and/or structures 230/231/232/234 can be formed separately, and thereafter coupled and/or connected together, while in some embodiments, one or more of the layers and/or structures 230/231/232/234 can be formed together using suitable techniques.

For example, in some embodiments, the layers and/or structures 230/231/232/234 can be formed separately, such as by extrusion, co-extrusion, interrupted layer co-extrusion (ILC), casting, molding, heat shrink techniques, fusing, winding, or the like, and thereafter coupled or connected together using suitable techniques, such as heat shrink techniques, friction fitting, mechanically fitting, chemically bonding, thermally bonding, welding (e.g., resistance, Rf, or laser welding), soldering, brazing, adhesive bonding, crimping, or the use of a connector member or material, or the like, or combinations thereof, to form the inner tubular member 224.

In some other embodiments, one or more of the layers and/or structures of the inner tubular member may be formed together at the same or similar times using suitable techniques, such as extrusion, co-extrusion, interrupted layer co-extrusion (ILC), or the like. In some other embodiments, one or more layers, for example the inner layer 234 and the reinforcing layer 231, can be formed and/or provided separately, and thereafter additional layers, for example layers 232 and 230, can be formed onto, over, or with the layers 231 and 234 by suitable techniques to form the inner tubular member 224.

The inner tubular member 224 may have a uniform stiffness, or may vary in stiffness along its length. For example, a gradual reduction in stiffness from the proximal end to the distal end thereof may be achieved, depending upon the desired characteristics. The gradual reduction in stiffness may be continuous or may be stepped, and may be achieved, for example, by varying the structure, such as the size, thickness, or other physical aspect of one or more of the layers 230/231/232/234, or for example, by varying the materials used in one or more of the layers 230/231/232/234. Such variability in characteristics and materials can be achieved, for example, by using techniques such as ILC, by fusing together separate extruded tubular segments, or in some cases, varying the characteristics and/or even the very presence or absence of certain structures and/or layers.

Referring to FIG. 7, the outer member 226 can also be a generally tubular member including a proximal region 236 having a proximal end 237 and a distal region 238 having a distal end 239. The outer member 226 can be disposed about at least a portion of the inner tubular member 224 at a location along the length of the shaft 212 between proximal end 218 and distal end 222. In the embodiment shown, the outer member 226 is disposed about the inner tubular member 226 along the proximal portion 216 of the shaft 212, but it should be understood that other locations are possible.

The length of the outer member 226 can also vary, depending upon, for example, the length of the shaft 212, the desired characteristics and functions of the catheter 210, and other such parameters. The outer member 226 may extend from a point within the distal portion 220 of the shaft 212 to a point within the proximal portion 216 of the shaft 212. In some embodiments, the outer member 226 has a length that allows it to be disposed over the majority of the length of the inner tubular member 224, and in some embodiments, is disposed about all but up to the distal most 15 cm or less, or 10 cm or less, or 5 cm or less of the inner tubular member 224 and/or all but the proximal most 15 cm or less, or 10 cm or less, or 5 cm or less of the inner tubular member 224. Outer member 226 may extend 50% or more, 75% or more, 90% or more, or 95% or more of the length of the shaft 212. In some embodiments, the length of the outer tubular member 226 can be in the range of about 1-299 centimeters or more, or in some embodiments in the range of about 19 cm-249 cm.

The outer member 226 defines a lumen 240 that can be adapted and/or configured to house or surround at least a portion of the inner tubular member 224. In some embodiments, the lumen 240 can have an inner diameter that is in the range of about 0.015 to about 0.06 inch in size, and in some embodiments, in the range of about 0.02 to about 0.035 inch in size. In some embodiments, the outer member 226 can have an outer diameter that is in the range of about 0.016 to about 0.07 inch in size, or in the range of about 0.02 to about 0.04 inch in size. It should be understood however, that these, and other dimensions provided herein, are by way of example only.

In at least some embodiments, the outer tubular member 226 can have an inner diameter that is greater than the outer diameter of the inner tubular member 224. As such, the outer tubular member 226 can be disposed about the inner tubular member 224 (i.e. a portion of the inner tubular member 224 is disposed within the lumen 240 of the outer member 226) such that a space or gap 242 is defined between at least a portion of the outer surface 225 of the inner tubular member 224 and the inner surface 227 of the outer member 226. In some embodiments, the space or gap 242 can be in the range of about 0.0002 to about 0.004 inch in size, and in some embodiments, in the range of about 0.0005 to about 0.003 inch in size. Again, it should be understood that these dimensions are provided by way of example only. However, in some embodiments the outer tubular member 226 may be substantially contiguous with the inner tubular member 224 such that no gap or space is formed between the inner tubular member 224 and the outer tubular member 226.

Typically, relatively large portions of the gap or space 242 remain open or unfilled by any other structure of the catheter 210 along a substantial portion of the length thereof, and in some cases along a substantial portion of the length of the outer member 226. For example, in some embodiments, 50% or more, 75% or more, 90% or more, or 95% or more of the gap or space 242 remains open and/or unfilled by any other structure of the catheter.

In some embodiments, the inner surface 227 of outer tubular member 226 or at least a distal portion of outer tubular member 226 is free of and/or unobstructed by any other structure of the device, thus the inner surface 227, or a portion thereof, may be exposed to the gap 242 defined between the outer surface 225 of the inner tubular member 224 and the inner surface 227 of the outer tubular member 226. Therefore, an exposed distal portion of the inner surface 227 of the outer tubular member 226 may be in direct contact with the bloodstream of a vessel, or other fluid medium, through apertures 244, as further described herein. Alternatively or additionally, at least a portion of the inner surface 227 may be coated with or covered by an electrically conductive structure. Thus, the portion of inner surface 227 including an electrically conductive structure may be in electrical communication with a bloodstream or other fluid medium within an anatomy through apertures 244.

In some embodiments, attachment points along the length of the outer member 226 may be used to attach to the inner tubular member 224. As a result, the gap or space 242 may be partially or totally filled at these attachment points, and as such, divided up into what may be considered multiple and/or a plurality of separate gaps or spaces that are unfilled. Additionally, other structures, such as coils, bands, braids, polymer layers, or the like, may fill portions of the gap or space 242. Even so, such multiple the gap or space 242, or the so defined multiple gaps or spaces 242 may still collectively extend along a substantial portion of the length of the outer tubular member 226 and remain overall substantially unfilled over the majority of the length thereof, for example, in percentages of the total length as given above. As such, the outer tubular member 226 can act to reinforce or impart desired properties, such as torsional and lateral rigidity, to the catheter shaft 212, and may allow at least the portion of the inner tubular member 224 surrounded by the gap or space 242 to be separate from, and in some cases bend and/or move laterally within, the lumen 240. Some examples of structure, methods, and techniques of coupling the outer member 226 to the inner tubular member 224 will be discussed in more detail below.

The outer member 226 can be adapted and/or configured to have a desired level of stiffness, torqueability, flexibility, and/or other characteristics. Those of skill in the art and others will recognize that the dimensions, structure, and materials of the outer member 226 are dictated primary by the desired characteristics, and the function of the final catheter 210, and that any of a broad range of the dimensions, structure, and materials can be used.

The desired stiffness, torquability, lateral flexibility, bendability or other such characteristics of the outer member 226 can be imparted or enhanced by the structure of the outer member 226. For example, the outer member 226 may include a thin wall tubular structure, including one or a plurality of apertures 244, such as grooves, cuts, slits, slots, or the like, formed in a portion of, or along the entire length of, the tubular outer member 226. Such structure may be desirable because it may allow outer member 226, or portions thereof, to have a desired level of lateral flexibility as well as have the ability to transmit torque and pushing forces from the proximal region 236 to the distal region 238. The apertures 244 can be formed in essentially any known way. For example, apertures 244 can be formed by methods such as micro-machining, saw-cutting, laser cutting, grinding, milling, casting, molding, chemically etching or treating, or other known methods, and the like. In some such embodiments, the structure of the outer member 226 is formed by cutting and/or removing portions of the tube to form apertures 244.

In some embodiments, the apertures 244 can completely penetrate the outer member 226 such that there is fluid communication between the lumen 240 and the exterior of the outer member 226 through the apertures 244. In some embodiments, the apertures 244 may only partially extend into the structure of the outer member 226, either on the interior or exterior surface thereof. Some other embodiments may include combinations of both complete and partial apertures 244 through the structure of the outer member 226. The shape and size of the apertures 244 can vary, for example, to achieve the desired characteristics. For example, the shape of apertures 244 can vary to include essentially any appropriate shape, such as square, round, rectangular, pill-shaped, oval, polygonal, elongate, irregular, or the like, and may include rounded or squared edges, and can be variable in length and width, and the like.

Additionally, the spacing, arrangement, and/or orientation of the apertures 244, or in some embodiments, associated spines or beams that may be formed, can be varied to achieve the desired characteristics. For example, the number or density of the apertures 244 along the length of the outer member 226 may be constant or may vary, depending upon the desired characteristics. For example, the number or proximity of apertures 244 to one another near one end of the outer member 226 may be high, while the number or proximity of slots to one another near the other end of the outer member 226, may be relatively low and/or non existent, or vice versa. For example, in the embodiment shown in FIGS. 5, 6, and 7, the distal region 238 of the outer member 226 includes a plurality of apertures 244 having a relatively high density relative to the plurality of apertures 244 located in the proximal region 236. As such, the distal region 238 can have a greater degree of lateral flexibility relative to the proximal region 236. The density of the apertures 244 can vary gradually or in a stepwise fashion over the length of the outer tubular member. And as suggested above, certain portions of the outer member 226 may not include any such apertures.

In some embodiments, the distal about 10 to about 50% of the total length of the outer member 226 can include apertures 244 defined therein at a relatively high density, while the proximal about 50 to about 90% of the total length of the outer member 226 include apertures 244 defined therein at a relatively low density, and/or is free of such apertures 244. For example, in some embodiments, the distal region 238 having a length in the range of about 30 to about 70 cm includes apertures 244 defined therein at a relatively high density to provide for relatively greater flexibility, while the remaining length in the proximal region 236 of the outer member 226 include apertures 244 defined therein at a relatively low density, and/or is free of such apertures 244, to provide for relatively greater stiffness. It should be understood however, that these, and other dimensions provided herein, are by way of example embodiments only, and that in other embodiments, the disposition of apertures 244 can vary greatly from the dimensions given, depending upon the desired characteristics and function of the device.

As suggested above, the apertures 244 may be formed such that one or more spines or beams 250 are formed in the tubular outer member 226. Such spines or beams 250 (FIG. 5) could include portions of the tubular member 226 that remain after the apertures 244 are formed in the body of the outer tubular member 226. Such spines or beams 250 may act to maintain a relatively high degree of torsional stiffness, while maintaining a desired level of lateral flexibility. In some embodiments, some adjacent apertures 244 can be formed such that they include portions that overlap with each other about the circumference of the tube. In other embodiments, some adjacent apertures 244 can be disposed such that they do not necessarily overlap with each other, but are disposed in a pattern that provides the desired degree of lateral flexibility. Additionally, the apertures 244 can be arranged along the length of, or about the circumference of, the outer member 226 to achieve desired properties. For example, the apertures 244 can be arranged in a symmetrical pattern, such as being disposed essentially equally on opposite sides about the circumference of the outer member 226, or equally spaced along the length of the outer member, or can be arranged in an increasing or decreasing density pattern, or can be arranged in a non-symmetric or irregular pattern.

Collectively, these figures and this description illustrate that changes in the arrangement, number, and configuration of slots may vary without departing from the scope of the invention. Some additional examples of shaft constructions and/or arrangements of cuts or slots formed in a tubular body are disclosed in U.S. Pat. No. 6,428,489 and in Published U.S. patent application Ser. Nos. 09/746,738 (Pub. No. US 2002/0013540), and 10/400,750 (Pub. No. US-2004-0193140-A1), all of which are incorporated herein by reference. Also, some additional examples of shaft constructions and/or arrangements of cuts or slots formed in a tubular body for use in a medical device are disclosed in U.S. patent application Ser. Nos. 10/375,493, and 10/400,750, which are also incorporated herein by reference.

In addition to, combination with, or as an alternative to the structure of the outer member 226, the materials selected for outer member 226 may also be chosen so that the outer member 226 may have the desired characteristics. The outer member 226 may be formed of any materials suitable for use, dependent upon the desired properties of the catheter 210. For example, outer member 226 may be formed of materials having a desired modulus of elasticity—given the structure used. Some examples of suitable materials include metals, metal alloys, polymers, or the like, or combinations or mixtures thereof.

Some examples of suitable metals and metal alloys include stainless steel, such as 304V, 304L, and 316L stainless steel; alloys including nickel-titanium alloy such as linear elastic or superelastic (i.e. pseudoelastic) nitinol; nickel-chromium alloy; nickel-chromium-iron alloy; cobalt alloy; tungsten or tungsten alloys; MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); hastelloy; monel 400; inconel 625; or the like; or other suitable material, or combinations or alloys thereof. Suitable metals and metal alloys may provide the outer tubular member 226 with a sufficient degree of conductivity. In some embodiments, it is desirable to use metals, or metal alloys that are suitable for metal joining techniques such as welding, soldering, brazing, crimping, friction fitting, adhesive bonding, etc. Some examples of suitable polymeric materials may include, but are not limited to: poly(L-lactide) (PLLA), poly(D,L-lactide) (PLA), polyglycolide (PGA), poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), polyethylene oxide (PEO), polydioxanone (PDS), polycaprolactone (PCL), polyhydroxylbutyrate (PHBT), poly(phosphazene), polyD,L-lactide-co-caprolactone) (PLA/PCL), poly(glycolide-co-caprolactone) (PGA/PCL), polyanhydrides (PAN), poly(ortho esters), poly(phoshate ester), poly(amino acid), poly(hydroxy butyrate), polyacrylate, polyacrylamid, poly(hydroxyethyl methacrylate), polyurethane, polysiloxane and their copolymers, or mixtures or combinations thereof. Some other potentially suitable polymer materials may include those listed above with reference to the inner tubular member 224.

As indicated above, some embodiments may include linear-elastic or super-elastic nitinol in various structures and/or components of the shaft 12 (e.g. outer tubular member 226, inner tubular member 224, etc.). The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL). In some embodiments, nitinol alloys can include in the range of about 45 to about 60 weight percent nickel, with the remainder being essentially titanium. It should be understood, however, that in other embodiments, the range of weight percent nickel and titanium, and or other trace elements may vary from these ranges. Within the family of commercially available nitinol alloys, are categories designated as “superelastic” (i.e. pseudoelastic) and “linear elastic” which, although similar in chemistry, exhibits distinct and useful mechanical properties.

In some embodiments, a superelastic alloy, for example a superelastic nitinol can be used to achieve desired properties. Such alloys typically display a substantial “superelastic plateau” or “flag region” in its stress/strain curve. Such alloys can be desirable in some embodiments because a suitable superelastic alloy will provide an outer member 226 that is exhibits some enhanced ability, relative to some other non-superelastic materials, of substantially recovering its shape without significant plastic deformation, upon the application and release of stress, for example, during placement of the catheter in the body.

In some other embodiments, a linear elastic alloy, for example a linear elastic nitinol can be used to achieve desired properties. For example, in some embodiments, certain linear elastic nitinol alloys can be generated by the application of cold work, directional stress, and/or heat treatment, such that the material fabricated does not display a substantial “superelastic plateau” or “flag region” in its stress/strain curve. Instead, in such embodiments, as recoverable strain increases, the stress continues to increase in a somewhat linear relationship until plastic deformation begins. In some embodiments, the linear elastic nickel-titanium alloy is an alloy that does not show any martensite/austenite phase changes that are detectable by DSC and DMTA analysis over a large temperature range. For example, in some embodiments, there are no martensite/austenite phase changes detectable by DSC and DMTA analysis in the range of about −60° C. to about 120° C. The mechanical bending properties of such material are therefore generally inert to the effect of temperature over a broad range of temperature. In some particular embodiments, the mechanical properties of the alloy at ambient or room temperature are substantially the same as the mechanical properties at body temperature. In some embodiments, the use of the linear elastic nickel-titanium alloy allows the outer member to exhibit superior “pushability” around tortuous anatomy. One example of a suitable nickel-titanium alloy exhibiting at least some linear elastic properties is FHP-NT alloy commercially available from Furukawa Techno Material Co. of Kanagawa, Japan. Additionally, some examples of suitable nickel-titanium alloy exhibiting at least some linear elastic properties include those disclosed in U.S. Pat. Nos. 5,238,004 and 6,508,803, which are incorporated herein by reference.

In some embodiments, the outer member 226, or other portions of the shaft 212, can be formed of a shape-memory material, for example a shape memory alloy such as a shape memory nitinol. In such embodiments, the shape memory effect can be used in the deployment or use of the catheter, for example in causing the outer member 226, or other portions of the shaft 212, to move from a first insertion configuration to a second use configuration, or, for example, for the outer member 226 to “remember” its desired shape after deformation to another shape.

For example, in some embodiments, the outer member 226 can include or be made of a shape memory alloy that is martensite at room temperature, and has a final austenite transition temperature (A_{f}) somewhere in the temperature range between room temperature and body temperature. For example, in some such embodiments, the shape memory alloy has a final austenite transition temperature in the range of about 25° C. and about 37° C. (e.g. about body temperature). In some such embodiments, it may be desirable that the final austenite transition temperature be at least slightly below body temperature, to ensure final transition at body temperature. This feature allows the outer member 226 to be inserted into the body of a patient in a martensitic state, and assume its preformed, austenitic shape when exposed to the higher body temperature within the anatomy, or at the target site. In this embodiment, deployment of the outer member 226 can be achieved by a shape memory effect—as the material warms, it undergoes a transition from martensite to austenite form, causing transformation of the outer member 226 from the first configuration to the second configuration.

In other example embodiments, the outer member 226 can include or be made of a shape-memory alloy that could have a transition temperature M_{d} (wherein M_{d}=highest temperature to strain-induced martensite) that is in the range of body temperature (e.g. about 37° C.) or greater, below which the alloy retains sufficient stress-induced martensitic property to allow placement of the outer member 226 at or above its final austenite transition temperature (A_{f}). In other words, this allows the catheter, including the outer member 226 in its preformed austenitic state, to be inserted and navigated in the anatomy, where the outer member 226 may be exposed to stress that may promote portions thereof to undergo stress-induced martensitic (SIM) transformation. Thereafter, the outer member 226 may recover its preformed, austenitic shape when released from the stress of navigation, at a temperature that may be substantially above the final austenite transition temperature without significant plastic, or otherwise permanent deformation. Additionally, in some such embodiments, the outer member 226 can be constrained, for example, in a delivery device, such as a guide catheter, in a stress-induced martensitic (SIM) state, and recover its preformed, austenitic shape when released from the constraints of the catheter, at a temperature that may be substantially above the final austenite transition temperature without significant plastic, or otherwise permanent deformation. In these embodiments, the final austenite temperature may be quite low, e.g., 4° C. or lower, or it may be up to room temperature or higher.

In yet other embodiments, the outer member 226 can include or be made of a shape memory alloy that is martensite at body temperature, and has a final austenite transition temperature (A_{f}) somewhere in the temperature range above body temperature. This feature allows the catheter including the outer member 26 to be navigated in a martensitic state, and maintain a martensitic state until exposed to a temperature higher than body temperature. The outer member 226 can then be heated to the necessary temperature above body temperature to make the transformation from martensite to austenite using an external heating means or mechanism. Such mechanisms may include the injection of heated fluid through the catheter, or other device, the use of electrical or other energy to heat the outer member 226, or other such techniques. In some such embodiments, the shape memory alloy has a final austenite transition temperature in the range of about 37° C. to about 45° C. It may be desirable that the final austenite transition temperature be at least slightly above body temperature, to ensure there is not final transition at body temperature. Some examples of nitinol cylindrical tubes having desired transition temperatures, as noted above, can be prepared according to known methods.

Referring to FIG. 7, the outer tubular member 226 may be connected to the inner tubular member 224 using any of a broad variety of suitable techniques, some examples of which may include adhesive bonding, friction fitting, mechanically fitting, crimping, chemically bonding, thermally bonding, welding (e.g., resistance, Rf, or laser welding), soldering, brazing, or the use of a connector member or material, or the like, or combinations thereof. As discussed above, in at least some embodiments, the outer tubular member 226 can be disposed about the inner tubular member 224 (i.e. a portion of the inner tubular member 224 is disposed within the lumen 240 of the outer member 226) such that a space or gap 242 is defined between at least a portion of the outer surface 225 of the inner tubular member 224 and the inner surface 227 of the outer member 226.

In FIG. 7, the outer member 226 is attached to the inner tubular member 224 at one or more proximal attachment point 253, one or more distal attachment point 259, and one or more intermediate attachment point 261. In some embodiments, such attachment points can be achieved, for example, using an adhesive material, for example, a cyanoacrylate, or other suitable type of adhesive. In at least some embodiments, only a relatively small portion of the outer member 226 is connected to the inner tubular member 224 at the attachment points. For example, the length of each individual bond joint, especially at the intermediate bond joints, may only be about 5 cm or less, or 3 cm or less, or 1 cm or less, or 0.5 cm or less. In some embodiments, where appropriate, the bonds extend under or within about five or fewer of the apertures 244, or three or even two or fewer of the apertures 244, along the length of the outer tubular member 226. Some embodiments may include a plurality of intermediate attachment point 261 spaced apart along the length of the shaft 212. In some embodiments, the distance between attachment points along the length of the shaft 212 may be in the range of about 5 cm and about 40 cm, or in the range of about 7 to about 30 cm, and may vary or be constant along the length of the shaft 212. For example, the spacing between attachment points may be closer together near the distal end of the shaft, and may be farther apart near the distal portion of the shaft 212.

As indicated above, the distal portion 220 of the shaft 212 can include a distal tip 228. The distal tip 228 can be a structure, assembly, construction and/or arrangement adapted and/or configured to provide characteristics such as shapability, flexibility, steerability, atraumatic characteristics, or the like, for example, to the distal portion and/or distal end of the shaft 212. A broad variety of distal tip constructions, configurations, and/or structures are generally know for use on medical devices, such as catheters, and may be used. In some embodiments, the distal tip 228 may be disposed at the distal portion 220 of the shaft 212, and may extend distally beyond other portions of the shaft 212.

In some embodiments, the distal tip 228 is simply portions of the shaft 212, and/or components thereof (e.g. the inner and/or outer tubular members 224/226) that include materials and/or structures to provide the desired characteristics. For example, in the embodiment shown in FIG. 7, the distal tip 228 can include and/or extend about the distal portion 220 of the inner tubular member 224. In this regard, the distal tip 228 may include the distal portion 220 of the inner tubular member 224, and may additionally include one or more additional layers and/or structures 252 disposed about the distal portion 220 of the inner tubular member 224. In other embodiments, however, the distal tip 228 may include structure and/or material that may be considered to be separate and distinct from other portions of the shaft, but that is connected to the distal portion of the shaft 212 to form the distal tip.

In FIG. 7, the layer 252 is disposed about the distal portion 220 of the inner tubular member 224. The layers 230, 232, and 234 of the inner tubular member 224 may include distal portions, for example 245 and 249, that include materials having desirable flexibility characteristics, for example, as discussed above. Additionally, the layer 252 may be made of or include any suitable material or structure, and may be disposed by any suitable process, the materials, structures, and processes varying with the particular application and characteristics desired. For example, in some embodiments, the one or more additional layers and/or structures may include a layer of polymer or other such material, or structures such as coils, braids, ribbons, wires, bands, of the like.

In this embodiment, the outer layer 252 may include and/or be made of a polymer material disposed about the distal portion 235 of the inner tubular member 224. For example, the outer layer 252 may include a flexible polymer material having a durometer in the range of about 5 D to about 35 D. Some examples of suitable polymers may include those discussed above with regard to the layers of the inner tubular member 224, with one example being a PEBA material, or the like. As can be appreciated, in some embodiments, the coil layer 231 extends partially into the distal tip 228, but ends and is spaced proximally from the distal end 222. In other embodiments, however, the coil 231, or other such reinforcing structure, or the like, may extend to the distal end 222. Additionally, it should be understood that one or more additional layers and/or constructions may be used in the distal tip 228.

The outer layer 252 may be sized appropriately so as to maintain a generally constant diameter in the transition between the outer member 226 and the outer layer 252, and may include a portion 265 that abuts and/or overlaps the distal end 239 of the outer member 226 to provide a smooth transition. Additionally, as in the embodiment shown, the outer tubular member 226 may include a recessed, or reduced diameter portion at the distal end 239 thereof, and the outer layer 252 may overlap with the recessed portion to provide for a smooth transition. In other embodiments, however, a tapered or step down transition may be provided.

The outer layer 252 can be constructed and/or disposed using any appropriate technique, for example, by extrusion, co-extrusion, interrupted layer co-extrusion (ILC), coating, heat shrink techniques, heat bonding, thermally bonding, casting, molding, fusing one or several segments of an outer layer material end-to-end, adhesive bonding, chemically bonding, crimping, friction fitting, mechanically fitting, or the like, or combinations thereof.

A lubricious, a hydrophilic, a protective, or other type of coating may be applied over portions of or the entire shaft 212. Hydrophobic coatings such as fluoropolymers provide a dry lubricity which improves catheter handling and device exchanges. Lubricious coatings can aid in insertion and steerability. Suitable lubricious polymers are well known in the art and may include silicone and the like, hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

In some embodiments, at least a distal portion of the outer tubular member 226 may be free of such a coating or may include an electrically conductive coating or covering. Thus, the outer surface of the outer tubular member 226 may be in electrical communication with a bloodstream or other fluid medium within an anatomy during use. For example, the outer surface of the outer tubular member 226 may have an exposed or unobstructed distal portion in direct contact with the bloodstream of a vessel, or another fluid medium with the anatomy, during use. Or, an electrically conductive coating or covering may provide an electrically conductive pathway between outer tubular member 226 and a fluid medium, such as a bloodstream, during use.

It should also be understood that in some embodiments, a degree of MRI compatibility can be imparted into shaft 212. For example, to enhance compatibility with Magnetic Resonance Imaging (MRI) machines, it may be desirable to construct portions of the outer tubular member 226, portions of the inner tubular member 224, or other portions of the shaft 212, in a manner, or use materials that would impart, a degree of MRI compatibility. For example, the lengths of relatively conductive structures within the shaft 212 may be limited to lengths that would not generate undue heat due to resonance waves created in such structures when under the influence of an MRI field generated by an MRI machine. Alternatively, or additionally, portions, or the entire shaft 212 may be made of a material that does not substantially distort the image and create substantial artifacts (artifacts are gaps in the image). Certain ferromagnetic materials, for example, may not be suitable because they may create artifacts in an MRI image. Additionally, all or portions of the shaft 212, may also be made of, impregnated with, plated or clad with, or otherwise include a material and/or structure that the MRI machine can image. Some materials that exhibit these characteristics include, for example, tungsten, Elgiloy, MP35N, nitinol, and the like, and others. Additionally, some structures including or made of such materials, such as marker bands, marker coils, rings, impregnated polymer sections, or the like, may be added to or included in the shaft 212. Those skilled in the art will recognize that these materials can vary widely without departing from the spirit of the invention.

Additionally, all or portions of the shaft 212, or components or layers thereof, may be made of, impregnated with, plated or clad with, or otherwise include a radiopaque material and/or structure to facilitate radiographic visualization. Radiopaque materials are understood to be materials capable of producing a relatively bright image on a fluoroscopy screen or another imaging technique during a medical procedure. This relatively bright image may aid the user of catheter 210 in determining its location. Some examples of radiopaque materials can include, but are not limited to, gold, platinum, palladium, tantalum, tungsten alloy, polymer material loaded with radiopaque filler, and the like.

For example, with reference to FIGS. 5-7, the inner tubular member 224 can include one or more radiopaque marker member 255 disposed in the distal portion 235 between the intermediate and outer layers 232/230, or at other positions and/or locations. Additionally, the outer tubular member 226 can include one or more marker member 255 disposed thereon. In the embodiment shown, the marker member 255 may be a tubular marker band, but it should be understood that other marker structures and arrangements, such as marker coils, rings, impregnated polymer sections, or the like, may be used, and may be disposed at locations along and/or within the shaft 212. Furthermore, the elongate shaft 212, or portions thereof, may be curved and/or shaped as desired, or be adapted and/or configured to be curved and/or shaped as desired, depending on the particular application.

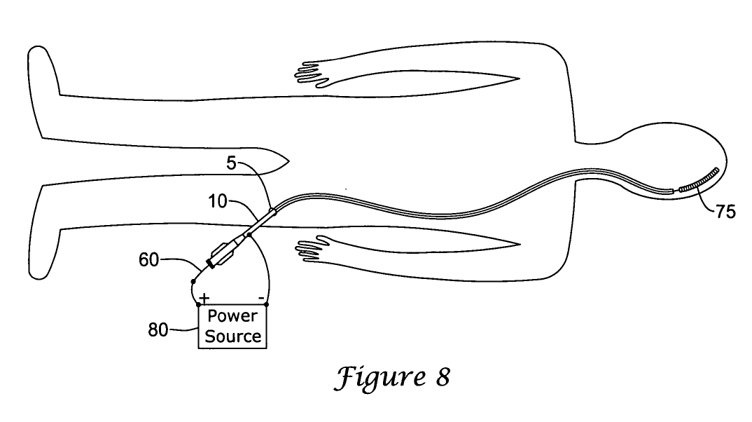

Now turning to a method of forming an occlusion in a vascular cavity in a vasculature of a body, FIG. 8 shows an exemplary navigational route to a target location. A vascular cavity, such as an aneurysm may be reached intravascularly by entering the vasculature of a body at a convenient location, such as the femoral artery. Catheter 10, which may be similar to catheter 210, may be inserted into the femoral artery at insertion point 5 and navigated to a vascular cavity, such as an intracranial aneurysm in the neurovascular region. In some embodiments, catheter 10 may be advanced through the vasculature distal of insertion point 5 a distance of 20 cm or more, 50 cm or more, 75 cm or more, or 100 cm or more. Catheter 10 may be positioned such that the distal end of catheter 10 is proximate the vascular cavity. A portion of catheter 10, including a proximal portion of the metallic tubular member 26 may be located external of the body of the patient proximal of insertion point 5 upon proper placement of the catheter 10. An occlusion member 75 detachably coupled to delivery wire 60 may be disposed in the lumen of catheter 10 and advanced to the vascular cavity.

A power supply 80 comprising an electrical current may be coupled to a proximal portion of the metallic tubular member 26 and a proximal portion of delivery wire 60. Power supply 80 may include a first terminal, such as a positive terminal, coupled to a proximal metallic portion of delivery wire 60, thus providing an electrically conductive pathway through delivery wire 60 to the intravascular target region. A second terminal, such as a negative terminal, of power supply 80 may be coupled to a proximal portion of metallic tubular member 26, thus providing an electrically conductive pathway through the metallic tubular member 26 to the intravascular target region. The bloodstream within the vessel, or another conductive fluid medium, may be used as an electrolyte to complete the electrolytic circuit. Thus, power supply 80 may be coupled to delivery wire 60 and metallic tubular member 26 such as to create an electrolytic cell near the intravascular target region.

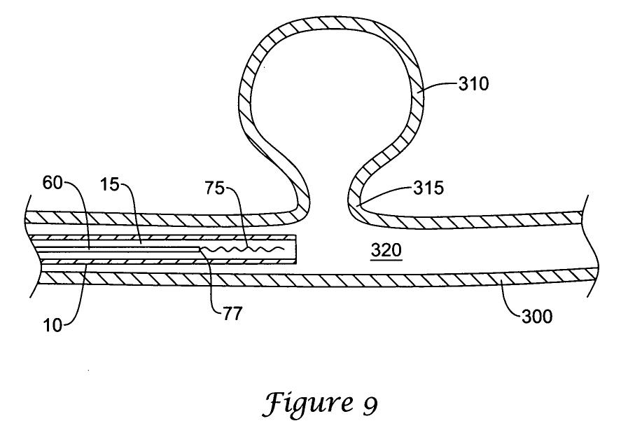

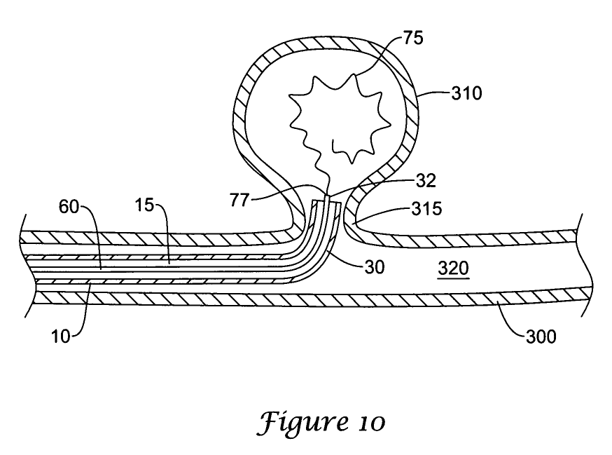

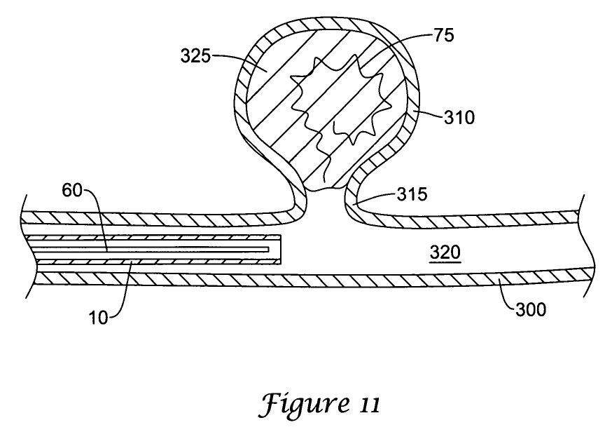

FIGS. 9-11 further illustrate an exemplary method of forming an occlusion within a vascular cavity, such as an intravascular aneurysm. As shown in FIG. 9, a distal portion of catheter 10 may be advanced through a vessel 300 to a location proximate the intravascular aneurysm 310. As shown in FIG. 10, the distal end of catheter 10 may be positioned to mate with, abut, or extend into opening 315 of aneurysm 310, or distal end of catheter 10 may remain in vessel 300 proximate opening 315. Using catheter 10 as a guide, occlusion member 75 and delivery wire 60 may be advanced through lumen 15 of catheter 10 to intravascular aneurysm 310. Delivery wire 60 may be further advanced distally, thus urging occlusion member 75 out of lumen 15 of catheter 10 and into the interior cavity of aneurysm 310. Unconstrained by catheter 10, occlusion member 75 may assume a coiled or expanded configuration and may be loosely deformed to the interior shape of the aneurysm 310. Thus, occlusion member 75 may be multiply folded or bent upon itself in the aneurysm to pack the interior of aneurysm 310.

After placement of the occlusion member 75 within the interior of aneurysm 310, a current may be applied to the delivery wire 60 from a power source (not shown) exterior of the body. Applying a current may initiate an electrolytic process at a distal portion of the assembly proximate the aneurysm 310. A distal portion of the metallic tubular member 26 may serve as a first electrode 30, which may act as a cathode, and sacrificial link 77 may serve as a second electrode 32, which may act as an anode. The portion of metallic tubular member 26 serving as first electrode 30 may be in electrical communication with bloodstream 320. For example, an exposed distal portion of metallic tubular member 26 may be in contact with the bloodstream 320 and located proximate the aneurysm 310, or a portion of metallic tubular member 26 serving as an electrode 30 may include an electrically conductive coating or covering in electrical communication with bloodstream 320. For instance, a distal portion of the metallic tubular member 26 may include an exposed portion or a coated or covered portion of the inner or outer surface of metallic tubular member in electrical communication with bloodstream 320. Alternatively or additionally, metallic tubular member 26 may be in electrical communication with another electrically conductive member serving as an electrode. The sacrificial link 75 serving as a second electrode 32 may also be in electrical communication, such as direct or indirect fluid contact, with the bloodstream 320. The bloodstream 320, or other fluid medium, may act as an electrolyte, allowing electrical current to pass through. Thus, a complete electrolytic cell is created intravascularly proximate the aneurysm 310. During the electrolytic process, oxidation takes place at the anode, which is a positive electrode, and reduction takes place at the cathode, which is a negative electrode. The first electrode 30, may be positioned proximate the second electrode 32 during an electrolytic process. For example, first electrode 30 may be positioned within about 20 cm or less, about 10 cm or less, about 5 cm or less, or about 1 cm or less of second electrode 32 during electrolysis. Through electrolysis, the sacrificial link 77 may be oxidized and dissipated, thus decoupling the occlusion member 75 from delivery wire 60.

As shown in FIG. 11, after the occlusion member is decoupled from the delivery wire 60, the delivery wire 60 and/or catheter 10 may be withdrawn from the vasculature. Thus, the occlusion member 75 is retained within the aneurysm 310. It may be necessary or desirable in some procedures to dispose one or more additional occlusion members 75 within an aneurysm 310 to more fully fill or pack the interior space of the aneurysm 310. In such instances, the catheter 10 may remain positioned in the vasculature while the delivery wire 60 is withdrawn and another delivery wire 60 including a second occlusion member 75 is advanced to the aneurysm 310. The electrolytic process may be repeated with the second or subsequent disposition of an occlusion member 75 within aneurysm 310. An aneurysm 310 packed with one or more occlusion members 75 may allow thrombus to form within the aneurysm 310, thus sufficiently occluding aneurysm 310. Hatched lines 325, shown in FIG. 11, illustrate occlusion of aneurysm 310.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A method of forming an occlusion within a vascular cavity in a vasculature of a body of a patient, the method comprising:
providing a catheter including a metallic tubular member having a proximal portion and a distal portion;
advancing the catheter through the vasculature such that the distal portion of the metallic tubular member is proximate the vascular cavity and the proximal portion of the metallic tubular member is external the body;
providing an elongate metallic delivery wire having a proximal portion and a distal portion, and an occlusion member detachably coupled to the distal portion of the delivery wire at a sacrificial link;
advancing the occlusion member and delivery wire through the catheter;
positioning the occlusion member within the vascular cavity such that the distal portion of the metallic tubular member is proximate the sacrificial link;
coupling a power source to the proximal portion of the metallic tubular member and the proximal portion of the delivery wire; and
applying an electrical current through the delivery wire and the metallic tubular member to electrolytically dissipate the sacrificial link such that the occlusion member is decoupled from the delivery wire, wherein a first electrode includes the distal portion of the metallic tubular member and a second electrode includes the sacrificial link.

2. The method of claim 1, wherein the first electrode is a cathode and the second electrode is an anode.

3. The method of claim 1, wherein the distal portion of the metallic tubular member is in electrical communication with an electrically conductive fluid within the body.

4. The method of claim 1, wherein the metallic tubular member includes an outer surface, and wherein at least a portion of the outer surface is in electrical communication with an electrically conductive fluid within the body.

5. The method of claim 1, wherein the metallic tubular member includes an inner surface, and wherein at least a portion of the inner surface is in electrical communication with an electrically conductive fluid within the body.

6. The method of claim 1, wherein the distal portion of the metallic tubular member is positioned within 20 cm or less of the sacrificial link.

7. The method of claim 1, wherein the distal portion of the metallic tubular member is positioned within 5 cm or less of the sacrificial link.

8. The method of claim 1, wherein the distal portion of the metallic tubular member is positioned within 1 cm or less of the sacrificial link.

9. The method of claim 1, wherein the catheter is advanced through the vasculature greater than 20 cm.

10. The method of claim 1, wherein the catheter is advanced through the vasculature greater than 50 cm.

11. The method of claim 1, wherein a distal end of the catheter abuts, mates with, or extends through an opening of the vascular cavity.

12. A method of depositing of a coil within an intracranial aneurysm in a vasculature of a body of a patient, the method comprising:
providing a microcatheter including a metallic tubular member having a proximal portion and a distal portion;
advancing the microcatheter through the vasculature such that the distal portion of the metallic tubular member is proximate the intracranial aneurysm and the proximal portion of the metallic tubular member is external the body;
providing an elongate metallic delivery wire having a proximal portion and a distal portion, and a coil detachably coupled to the distal portion of the delivery wire at a sacrificial link;
advancing the coil and delivery wire through the microcatheter;
positioning the coil within the intracranial aneurysm such that the distal portion of the metallic tubular member is proximate the sacrificial link;
coupling a power source to the proximal portion of the metallic tubular member and the proximal portion of the delivery wire; and
applying an electrical current through the delivery wire and the metallic tubular member to electrolytically dissipate the sacrificial link such that the coil is decoupled from the delivery wire, wherein a first electrode includes the distal portion of the metallic tubular member and a second electrode includes the sacrificial link.

13. The method of claim 12, wherein the first electrode is a cathode and the second electrode is an anode.

14. The method of claim 12, wherein the distal portion of the metallic tubular member is in electrical communication with a bloodstream.

15. The method of claim 12, wherein the distal portion of the metallic tubular member is positioned within 10 cm or less of the sacrificial link.

16. The method of claim 12, wherein the distal portion of the metallic tubular member is positioned within 5 cm or less of the sacrificial link.

17. A method of forming an occlusion within a vascular cavity, the method comprising:
providing a catheter having a proximal end, a distal end, a length and a lumen extending therethrough, the catheter including a metallic tubular member extending a majority of the length of the catheter, the metallic tubular member having a proximal portion and a distal portion;
advancing the catheter through a vasculature such that the distal end of the catheter is proximate the vascular cavity;
providing an elongate metallic delivery wire having a proximal portion and a distal portion, and an occlusion member detachably coupled to the distal portion of the delivery wire;
advancing the occlusion member and delivery wire through the lumen of the catheter to the vascular cavity;
positioning the occlusion member within the vascular cavity, such that the distal portion of the metallic tubular member is proximate the occlusion member;
coupling an electrical power source to the proximal portion of the metallic tubular member and the proximal portion of the delivery wire; and
applying an electrical current through the delivery wire and the metallic tubular member to electrolytically decouple the occlusion member from the delivery wire, wherein a first electrode includes the distal portion of the metallic tubular member and a second electrode includes the distal portion of the delivery wire.

18. The method of claim 17, wherein the occlusion member extends distal of the distal portion of the delivery wire.

19. The method of claim 17, wherein the first electrode is a cathode and the second electrode is an anode.

20. The method of claim 17, wherein the distal portion of the metallic tubular member is an exposed distal portion.

21. The method of claim 17, wherein the distance between the distal portion of the metallic tubular member and the occlusion member when the occlusion member is positioned within the vascular cavity is less than 50 cm.

22. The method of claim 17, wherein the distance between the distal portion of the metallic tubular member and the occlusion member when the occlusion member is positioned within the vascular cavity is less than 20 cm.

23. The method of claim 17, wherein the distance between the distal portion of the metallic tubular member and the occlusion member when the occlusion member is positioned within the vascular cavity is less than 5 cm.

24. The method of claim 17, wherein the distance between the distal portion of the metallic tubular member and the occlusion member when the occlusion member is positioned within the vascular cavity is less than 1 cm.

25. The method of claim 17, wherein the catheter is advanced through the vasculature greater than 20 cm.

26. The method of claim 17, wherein the catheter is advanced through the vasculature greater than 50 cm.

27. The method of claim 17, wherein the distal end of the catheter abuts, mates with, or extends through an opening of the vascular cavity.

28. An assembly for electrolytically disposing a coil in a vascular cavity, the assembly comprising:
a cathode comprising an exposed distal portion of a metallic tubular member, the metallic tubular member extending from a proximal portion to a distal portion of a catheter and defining a catheter lumen, the metallic tubular member providing an electrically conductive pathway during use;
an anode comprising a sacrificial link between a metallic delivery wire and a coil, the delivery wire providing an electrically conductive pathway during use;
wherein the delivery wire is positioned in the catheter lumen such that the anode is proximate the cathode during use in a vascular region; and
a current source coupled to a proximal portion of the metallic tubular member and a proximal portion of the delivery wire, wherein during use the current source provides an electrolytic current through the assembly to dissipate the sacrificial link such that the coil is decoupled from the delivery wire.

29. The assembly of claim 28, wherein during use the anode is positioned less than 20 cm from the cathode.

30. The assembly of claim 28, wherein during use the anode is positioned less than 5 cm from the cathode.

31. The assembly of claim 28, wherein the metallic tubular member comprises a plurality of slots and beams.

32. An assembly for forming an occlusion in a vascular cavity, the assembly comprising:
a catheter having a proximal end, a distal end, a length and a lumen extending therethrough, the catheter including a metallic tubular member extending a majority of the length of the catheter, the metallic tubular member having a proximal portion and a distal portion, wherein the metallic tubular member provides an electrically conductive pathway during use;
an elongate metallic delivery wire having a proximal portion and a distal portion, wherein the delivery wire provides an electrically conductive pathway during use;
an occlusion member detachably coupled to the distal portion of the delivery wire at a sacrificial link;
wherein during use the delivery wire is disposed through the lumen of the catheter such that the distal portion of the delivery wire is proximate the distal portion of the metallic tubular member when the occlusion member is positioned within the vascular cavity;
a current source coupled to the proximal portion of the metallic tubular member and the proximal portion of the delivery wire, wherein the current source provides an electrolytic current through the metallic tubular member and the delivery wire to initiate electrolysis at the sacrificial link; and
wherein the distal portion of the metallic tubular member serves as a cathode and the sacrificial link serves as an anode during electrolysis.

33. The assembly of claim 32, wherein the length of the catheter is greater than 20 cm.

34. The assembly of claim 32, wherein the length of the catheter is greater than 50 cm.

35. The assembly of claim 32, wherein the length of the catheter is greater than 100 cm.

36. The assembly of claim 32, wherein the occlusion member is a coil.

37. The assembly of claim 32, wherein the occlusion member comprises platinum.

38. The assembly of claim 32, wherein the delivery wire comprises stainless steel.

39. The assembly of claim 32, wherein the metallic tubular member comprises stainless steel, a stainless steel alloy, or a nickel-titanium alloy.

40. The assembly of claim 32, wherein the current source includes a direct current source having a positive terminal and a negative terminal, wherein the positive terminal is connected to the delivery wire and the negative terminal is connected to the metallic tubular member of the catheter.

41. The assembly of claim 32, wherein the metallic tubular member has an outer surface, and wherein at least a portion of the outer surface is in electrical communication with an electrically conductive fluid within the body.

42. The assembly of claim 32, wherein the metallic tubular member has an inner surface, and wherein at least a portion of the inner surface is in electrical communication with an electrically conductive fluid within the body.

43. The assembly of claim 32, wherein the metallic tubular member comprises a plurality of slots and beams.

44. A kit for depositing a coil within an aneurysm in a vasculature of a body of a patient, the kit comprising:
a catheter having a proximal end, a distal end, a length and a lumen extending therethrough, the catheter including a metallic tubular member extending a majority of the length of the catheter, the metallic tubular member having a proximal portion and a distal portion, wherein the distal portion acts as a cathode during use;
an elongate metallic delivery wire having a proximal portion and a distal portion;
a coil detachably coupled to the distal portion of the delivery wire at a sacrificial link, wherein the sacrificial link acts as an anode during use; and
a power supply coupled to the proximal portion of the metallic delivery wire and the proximal portion of the metallic tubular member;
wherein the kit forms an intravascular electrolytic cell during use.

45. The kit of claim 44, wherein the catheter has a length greater than 20 cm

46. The kit of claim 44, wherein the catheter has a length greater than 50 cm.

47. The kit of claim 44, wherein the catheter has a length greater than 100 cm.

48. The kit of claim 44, wherein the power supply is a direct current power supply having a positive terminal and a negative terminal, and wherein the positive terminal is coupled to the metallic delivery wire and the negative terminal is coupled to the metallic tubular member.

49. The kit of claim 44, wherein the metallic tubular member includes a plurality of slots and beams.

50. An electrolytic cell of a medical device assembly, the electrolytic cell comprising:
a cathode comprising a distal portion of a metallic tubular member exposed to a bloodstream, the metallic tubular member extending from a proximal portion to a distal portion of a catheter and defining a catheter lumen, the metallic tubular member providing an electrically conductive pathway during use;
an anode comprising a sacrificial link exposed to a bloodstream, the sacrificial link joining a metallic delivery wire and a coil, the delivery wire providing an electrically conductive pathway during use;
wherein the delivery wire is positioned in the catheter lumen such that the anode is proximate the cathode; and
a current source coupled to a proximal portion of the metallic tubular member and a proximal portion of the delivery wire.
